(19) United States
(12) Patent Application Publication
(10) Pub. No.: US 2018/0155497 A1
(43) Pub. Date: Jun. 7, 2018

(54) Renewably Derived Polyamides and Methods of Making the Same

(71) Applicant: Elevance Renewable Sciences, Inc., Woodridge, IL (US)

(72) Inventors: Jordan R. Quinn, Mundelein, IL (US); Paul A. Bertin, Western Springs, IL (US)

(73) Assignee: Elevance Renewable Sciences, Inc., Woodridge, IL (US)

(21) Appl. No.: 15/787,300

(22) Filed: Oct. 18, 2017

Related U.S. Application Data
(60) Provisional application No. 62/412,709, filed on Oct. 25, 2016.

(51) Int. Cl. C08G 69/08; C07C 227/06; C07C 51/09; C07C 51/363

(52) U.S. Cl. CPC C08G 69/08; C07C 51/363; C07C 51/09; C07C 227/06

(57) ABSTRACT

Methods of making polyamides from renewable materials, such as natural oils, are generally disclosed herein. In some embodiments, the polyamides are nylon-10. In some such embodiments, nylon-10 is made by polymerizing 10-aminodecanoic acid, or esters thereof. In some further such embodiments, the 10-aminodecanoic acid monomers (or esters thereof) are derived from natural oils via the metathesis of unsaturated fatty acid moieties of the natural oil.

### CROSS-REFERENCE TO RELATED APPLICATIONS

The present application claims the benefit of priority of U.S. Provisional Application No. 62/412,709, filed Oct. 25, 2016, which is hereby incorporated by reference as though set forth herein in its entirety.

### TECHNICAL FIELD

Methods of making polyamides from renewable materials, such as natural oils, are generally disclosed herein. In some embodiments, the polyamides are nylon-10. In some such embodiments, nylon-10 is made by polymerizing 10-aminodecanoic acid, or esters thereof. In some further such embodiments, the 10-aminodecanoic acid monomers (or esters thereof) are derived from natural oils via the metathesis of unsaturated fatty acid moieties of the natural oil, e.g., from their reaction with short-chain alpha-olefins.

### BACKGROUND

Polyamide homopolymers, such as nylon 12, have certain desirable properties and have found wide use in various industries. Such longer-chain polyamides tend to have properties that lie between those of short-chain polyamides, such as nylon 6,6 or nylon 6, and those of polyolefins. But the availability of these long-chain polyamides is limited due to the cost of making the monomers from which they are made.

For example, nylon 12 is generally made either through the homopolymerization of ω-aminolauric acid or through the ring-opening polymerization of laurolactam at high temperatures. In both cases, the starting material relies on the availability of a starting material that can be expensive and whose supply is not predictable. For example, in 2012, a worldwide shortage of nylon 12 occurred due to an incident at a single plant in Germany that manufactured a precursor to the starting material.

Moreover, the most commonly used process for making nylon 12, the ring-opening polymerization of laurolactam, uses cyclododecatriene to make the laurolactam monomer. Cyclododecatriene is derived from the trimerization of butadiene, which is typically derived as a byproduct of petroleum cracking, e.g., through steam cracking. Therefore, as the end-users increasingly move towards trying to increase the “green” content of their products, the use of such petroleum-derived materials is becoming increasingly undesirable in the marketplace.

Therefore, there is a continuing need to develop materials that may serve as suitable replacements for long-chain polyamides and thereby overcome one or more of the aforementioned shortcomings of nylon 12.

### SUMMARY

The present disclosure overcomes one or more of the aforementioned shortcomings by providing a process for making a long-chain polyamide, nylon 10, by a process in which most of the carbons in the resulting polyamide are derived from renewable sources.

In a first aspect, the disclosure provides methods of making a polyamide from a natural oil, the method comprising: providing 9-decenoic acid, wherein providing 9-decenoic acid comprises deriving 9-decenoic acid from a natural oil composition; reacting 9-decenoic acid with a brominating agent to form 10-bromodecanoic acid; reacting 10-bromodecanoic acid with an aminating agent to form 10-aminodecanoic acid; and polymerizing 10-aminodecanoic acid to form a nylon-10 polymer.

In a second aspect, the disclosure provides methods of making a polyamide from a natural oil, the method comprising: providing C₁₋₈ alkyl esters of 9-decenoic acid, wherein providing C₁₋₈ alkyl esters of 9-decenoic acid comprises deriving C₁₋₈ alkyl esters of 9-decenoic acid from a natural oil composition; reacting esters of C₁₋₈ alkyl 9-decenoic acid with a brominating agent to form C₁₋₈ alkyl esters of 10-bromodecanoic acid; reacting C₁₋₈ alkyl esters of 10-bromodecanoic acid with an aminating agent to form C₁₋₈ alkyl esters of 10-aminodecanoic acid; converting the C₁₋₈ alkyl esters of 10-aminodecanoic acid to 10-aminodecanoic acid; and polymerizing 10-aminodecanoic acid to form a nylon-10 polymer.

In a third aspect, the disclosure provides methods of making a polyamide from a natural oil, the method comprising: providing C₁₋₈ alkyl esters of 9-decenoic acid, wherein providing C₁₋₈ alkyl esters of 9-decenoic acid comprises deriving C₁₋₈ alkyl esters of 9-decenoic acid from a natural oil composition; reacting esters of C₁₋₈ alkyl 9-decenoic acid with a brominating agent to form C₁₋₈ alkyl esters of 10-bromodecanoic acid; reacting C₁₋₈ alkyl esters of 10-bromodecanoic acid with an aminating agent to form C₁₋₈ alkyl esters of 10-aminodecanoic acid; and polymerizing C₁₋₈ alkyl esters of 10-aminodecanoic acid to form a nylon-10 polymer.

Further aspects and embodiments are provided in the foregoing drawings, detailed description and claims.

### BRIEF DESCRIPTION OF THE DRAWINGS

Not applicable.

### DETAILED DESCRIPTION

The following description recites various aspects and embodiments of the inventions disclosed herein. No particular embodiment is intended to define the scope of the invention. Rather, the embodiments provide non-limiting examples of various compositions, and methods that are included within the scope of the claimed inventions. The description is to be read from the perspective of one of ordinary skill in the art. Therefore, information that is well known to the ordinarily skilled artisan is not necessarily included.

### Definitions

The following terms and phrases have the meanings indicated below, unless otherwise provided herein. This disclosure may employ other terms and phrases not expressly defined herein. Such other terms and phrases shall have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure, and are not meant to be limiting in any fashion. Nor do these phrases indicate any kind of preference for the disclosed embodiment.

As used herein, “polymer” refers to a substance having a chemical structure that includes the multiple repetition of constitutional units formed from substances of comparatively low relative molecular mass relative to the molecular mass of the polymer. The term “polymer” includes soluble and/or fusible molecules having chains of repeat units, and also includes insoluble and infusible networks. As used herein, the term “polymer” can include oligomeric materials, which have only a few (e.g., 3-100) constitutional units.

As used herein, “natural oil” refers to oils obtained from plants or animal sources. The terms also include modified plant or animal sources (e.g., genetically modified plant or animal sources), unless indicated otherwise. Examples of natural oils include, but are not limited to, vegetable oils, algae oils, fish oils, animal fats, tall oils, derivatives of these oils, combinations of any of these oils, and the like. Representative non-limiting examples of vegetable oils include rapeseed oil (canola oil), coconut oil, corn oil, cottonseed oil, olive oil, palm oil, peanut oil, safflower oil, sesame oil, soybean oil, sunflower oil, linseed oil, palm kernel oil, tung oil, jatropha oil, mustard seed oil, pennycress oil, camelina oil, hempseed oil, and castor oil. Representative non-limiting examples of animal fats include lard, tallow, poultry fat, yellow grease, and fish oil. Tall oils are by-products of wood pulp manufacture. In some embodiments, the natural oil or natural oil feedstock comprises one or more unsaturated glycerides (e.g., unsaturated triglycerides). In some such embodiments, the natural oil comprises at least 50% by weight, or at least 60% by weight, or at least 70% by weight, or at least 80% by weight, or at least 90% by weight, or at least 95% by weight, or at least 97% by weight, or at least 99% by weight of one or more unsaturated triglycerides, based on the total weight of the natural oil.

The term “unsaturated natural fatty acid” refers to an unsaturated fatty acid obtained from a natural oil (defined above). Analogously, the term “unsaturated natural fatty acid ester” refers to esters of such unsaturated fatty acids, such as glyceryl esters (e.g., monoacylglycerides, diacylglycerides, and triacylglyceriedes), alkyl esters, and the like.

As used herein, “metathesis” refers to olefin metathesis. As used herein, “metathesis catalyst” includes any catalyst or catalyst system that catalyzes an olefin metathesis reaction.

As used herein, “metathesize” or “metathesizing” refer to the reacting of a feedstock in the presence of a metathesis catalyst to form a “metathesized product” comprising new olefinic compounds, i.e., “metathesized” compounds. Metathesizing is not limited to any particular type of olefin metathesis, and may refer to cross-metathesis (i.e., co-metathesis), self-metathesis, ring-opening metathesis, ring-opening metathesis polymerizations (“ROMP”), ring-closing metathesis (“RCM”), and acyclic diene metathesis (“ADMET”). In some embodiments, metathesizing refers to reacting two triglycerides present in a natural feedstock (self-metathesis) in the presence of a metathesis catalyst, wherein each triglyceride has an unsaturated carbon-carbon double bond, thereby forming a new mixture of olefins and esters which may include a triglyceride dimer. Such triglyceride dimers may have more than one olefinic bond, thus higher oligomers also may form. Additionally, in some other embodiments, metathesizing may refer to reacting an olefin, such as ethylene, and a triglyceride in a natural feedstock having at least one unsaturated carbon-carbon double bond, thereby forming new olefinic molecules as well as new ester molecules (cross-metathesis).

As used herein, “olefin” or “olefins” refer to compounds having at least one unsaturated carbon-carbon double bond. In certain embodiments, the term “olefins” refers to a group of unsaturated carbon-carbon double bond compounds with different carbon lengths. Unless noted otherwise, the terms “olefin” or “olefins” encompasses “polyunsaturated olefins” or “poly-olefins,” which have more than one carbon-carbon double bond. As used herein, the term “monounsaturated olefins” or “mono-olefins” refers to compounds having only one carbon-carbon double bond. A compound having a terminal carbon-carbon double bond can be referred to as a “terminal olefin” or an “alpha-olefin,” while an olefin having a non-terminal carbon-carbon double bond can be referred to as an “internal olefin.” In some embodiments, the alpha-olefin is a terminal alkene, which is an alkene (as defined below) having a terminal carbon-carbon double bond. Additional carbon-carbon double bonds can be present.

The number of carbon atoms in any group or compound can be represented by the terms: “C_{z}”, which refers to a group of compound having z carbon atoms; and “C_{x-y}”, which refers to a group or compound containing from x to y, inclusive, carbon atoms. For example, “C₁₋₆ alkyl” represents an alkyl chain having from 1 to 6 carbon atoms and, for example, includes, but is not limited to, methyl, ethyl, n-propyl, isopropyl, isobutyl, n-butyl, sec-butyl, tert-butyl, isopentyl, n-pentyl, neopentyl, and n-hexyl. As a further example, a “C₄₋₁₀ alkene” refers to an alkene molecule having from 4 to 10 carbon atoms, and, for example, includes, but is not limited to, 1-butene, 2-butene, isobutene, 1-pentene, 1-hexene, 3-hexene, 1-heptene, 3-heptene, 1-octene, 4-octene, 1-nonene, 4-nonene, and 1-decene.

As used herein, the term “short-chain alpha olefin” refers to any one or combination of unsaturated straight, branched, or cyclic hydrocarbons in the C₂₋₁₄ range, or the C₂₋₁₂ range, or the C₂₋₁₀ range, or the C₂₋₈ range, having at least one terminal carbon-carbon double bond. Such olefins also include dienes or trienes. Examples of short-chain alpha olefins include, but are not limited to: ethylene, propylene, 1-butene, isobutene, 1-pentene, 3-methyl-1-butene, 1,4-pentadiene, 1-hexene, 2-methyl-1-pentene, 3-methyl-1-pentene, and 4-methyl-1-pentene.

As used herein, “alkyl” refers to a straight or branched chain saturated hydrocarbon having 1 to 30 carbon atoms, which may be optionally substituted, as herein further described, with multiple degrees of substitution being allowed. Examples of “alkyl,” as used herein, include, but are not limited to, methyl, ethyl, n-propyl, isopropyl, isobutyl, n-butyl, sec-butyl, tert-butyl, isopentyl, n-pentyl, neopentyl, n-hexyl, and 2-ethylhexyl. The number of carbon atoms in an alkyl group is represented by the phrase “C_{x-y} alkyl,” which refers to an alkyl group, as herein defined, containing from x to y, inclusive, carbon atoms. Thus, “C₁₋₆ alkyl” represents an alkyl chain having from 1 to 6 carbon atoms and, for example, includes, but is not limited to, methyl, ethyl, n-propyl, isopropyl, isobutyl, n-butyl, sec-butyl, tert-butyl, isopentyl, n-pentyl, neopentyl, and n-hexyl. In some instances, the “alkyl” group can be divalent, in which case the group can alternatively be referred to as an “alkylene” group.

As used herein, “mix” or “mixed” or “mixture” refers broadly to any combining of two or more compositions. The two or more compositions need not have the same physical state; thus, solids can be “mixed” with liquids, e.g., to form a slurry, suspension, or solution. Further, these terms do not require any degree of homogeneity or uniformity of composition. This, such “mixtures” can be homogeneous or heterogeneous, or can be uniform or non-uniform. Further, the terms do not require the use of any particular equipment to carry out the mixing, such as an industrial mixer.

As used herein, “optionally” means that the subsequently described event(s) may or may not occur. In some embodiments, the optional event does not occur. In some other embodiments, the optional event does occur one or more times.

As used herein, “comprise” or “comprises” or “comprising” or “comprised of” refer to groups that are open, meaning that the group can include additional members in addition to those expressly recited. For example, the phrase, “comprises A” means that A must be present, but that other members can be present too. The terms “include,” “have,” and “composed of” and their grammatical variants have the same meaning. In contrast, “consist of” or “consists of” or “consisting of” refer to groups that are closed. For example, the phrase “consists of A” means that A and only A is present.

As used herein, “or” is to be given its broadest reasonable interpretation, and is not to be limited to an either/or construction. Thus, the phrase “comprising A or B” means that A can be present and not B, or that B is present and not A, or that A and B are both present. Further, if A, for example, defines a class that can have multiple members, e.g., A₁ and A₂, then one or more members of the class can be present concurrently.

In some instances herein, organic compounds are described using the “line structure” methodology, where chemical bonds are indicated by a line, where the carbon atoms are not expressly labeled, and where the hydrogen atoms covalently bound to carbon (or the C—H bonds) are not shown at all. For example, by that convention, the formula

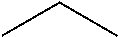

represents n-propane. In some instances herein, a squiggly bond is used to show the compound can have any one of two or more isomers. For example, the structure

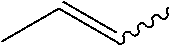

can refer to (E)-2-butene or (Z)-2-butene. The same is true when olefinic structures are drawn that are ambiguous as to which isomer is referred to. For example, CH₃—CH═CH—CH₃ can refer to (E)-2-butene or (Z)-2-butene.

As used herein, the various functional groups represented will be understood to have a point of attachment at the functional group having the hyphen or dash (-) or an asterisk (*). In other words, in the case of —CH₂CH₂CH₃, it will be understood that the point of attachment is the CH₂ group at the far left. If a group is recited without an asterisk or a dash, then the attachment point is indicated by the plain and ordinary meaning of the recited group.

As used herein, multi-atom bivalent species are to be read from left to right. For example, if the specification or claims recite A-D-E and D is defined as —OC(O)—, the resulting group with D replaced is: A-OC(O)-E and not A-C(O)O-E.

Other terms are defined in other portions of this description, even though not included in this subsection.

Methods of Making Polyamides by Aqueous Condensation from ω-Amino Acid

In at least one aspect, the disclosure provides methods of making a polyamide from a natural oil, the method comprising: providing 9-decenoic acid; reacting 9-decenoic acid with a brominating agent to form 10-bromodecanoic acid; reacting 10-bromodecanoic acid with an aminating agent to form 10-aminodecanoic acid; and polymerizing 10-aminodecanoic acid to form a nylon-10 polymer.

In some embodiments, providing 9-decenoic acid comprises deriving 9-decenoic acid from a natural oil composition, such as a natural oil (as defined above) or any composition comprising a natural oil. Deriving the 9-decenoic acid from a natural oil can be accomplished by any suitable means. For example, in some embodiments, deriving 9-decenoic acid from a natural oil comprises: providing a natural oil composition comprising unsaturated natural fatty acid esters; reacting the unsaturated natural fatty acid esters with a short-chain alpha-olefin in the presence of a metathesis catalyst to form 9-decenoic acid esters and 1-decene; and converting the 9-decenoic acid esters to 9-decenoic acid.

The esters set forth in the foregoing embodiments can be any suitable esters, e.g., esters made from any suitable alcohol. In some embodiments, the unsaturated natural fatty acid esters are C₁₋₈ alkyl esters of unsaturated natural fatty acids, and wherein the 9-decenoic acid esters are C₁₋₈ alkyl esters of 9-decenoic acid. In some further such embodiments, the unsaturated natural fatty acid esters are methyl esters of unsaturated natural fatty acids, and wherein the 9-decenoic acid esters are methyl esters of 9-decenoic acid. In some other embodiments, the unsaturated natural fatty acid esters are glyceryl esters of unsaturated natural fatty acids, and wherein the 9-decenoic acid esters are glyceryl esters of 9-decenoic acid. In such embodiments, any suitable glyceryl ester can be used, including monoacylglycerides, diacylglycerides, and triacylglycerides. In some such embodiments, the esters are triacylglycerides, such as those commonly found in natural oils.

Any suitable unsaturated natural fatty acids can be used in the methods disclosed herein. In some embodiments, the unsaturated natural fatty acids are unsaturated fatty acids having a carbon-carbon double bond between the ninth and tenth carbon atoms counting from the ester group (including the carbon in the carbonyl of the ester). In some embodiments, the unsaturated natural fatty acids are selected from the group consisting of myristoleic acid, palmitoleic acid, oleic acid, elaidic acid, linoleic acid, linoelaidic acid, and α-linolenic acid. In some further embodiments, the unsaturated natural fatty acids are selected from the group consisting of oleic acid, linoleic acid, and α-linolenic acid.

Converting the 9-decenoic acid esters to 9-decenoic acid can be carried out by any suitable means. In some embodiments, converting the 9-decenoic acid esters to 9-decenoic acid comprises hydrolyzing the 9-decenoic acid esters to form 9-decenoic acid, e.g., using standard hydrolysis conditions. In some other embodiments, converting the 9-decenoic acid esters to 9-decenoic acid comprises: saponifying the 9-decenoic acid esters to form 9-decenoate anions; and acidifying the 9-decenoate anions to form 9-decenoic acid. In some other embodiments, where the 9-decenoic acid is an ester besides an alkyl ester (e.g., a glyceryl ester), the conversion to the acid does not occur directly, but first involves a transesterification to an alkyl ester, followed by the converting of the alkyl ester to the acid. For example, in some embodiments, converting the 9-decenoic acid esters to 9-decenoic acid comprises: reacting the glyceryl esters of 9-decenoic acid with a C₁₋₈ monohydric alkanol to form C₁₋₈ esters of 9-decenoic acid; and converting the C₁₋₈ esters of 9-decenoic acid to 9-decenoic acid. In such processes, any suitable C₁₋₈ monohydric alkanol (i.e., R—OH, where R is a C₁₋₈ alkyl) can be used. Non-limiting examples include methanol, ethanol, propanol, isopropanol, butanol, sec-butanol, tert-butanol, pentanol, neopentanol, hexanol, heptanol, octanol, and 2-ethylhexanol. In some embodiments, the C₁₋₈ monohydric alkanol is methanol.

In any of the aforementioned embodiments, reacting the unsaturated natural fatty acid esters with a short-chain alpha-olefin in the presence of a metathesis catalyst can be carried out by any suitable means. Principles of metathesis are discussed in greater detail in a subsequent subsection, and can be applied here. Any suitable short-chain alpha-olefin can be used. For example, in some embodiments, the short-chain alpha olefin is selected from the group consisting of: ethylene, propylene, 1-butene, isobutene, 1-pentene, 1-hexene, 1-heptene, 1-octene, and 1-nonene. In some further such embodiments, the short-chain alpha olefin is selected from the group consisting of: ethylene, propylene, and 1-butene. In some further such embodiments, the short-chain alpha olefin is selected from the group consisting of: ethylene or 1-butene.

The bromination can be carried by any suitable means. In some embodiments, the brominating agent is hydrobromic acid. Known methods of hydrobromination can be suitably modified to achieve the desired result, depending on the relevant scale.

The amination can be carried by any suitable means. In some embodiments, the aminating agent is ammonia. Known methods of reacting an alkyl bromide with ammonia can be suitably modified to achieve the desired result, depending on the relevant scale.

Methods of Making Polyamides by Aqueous Condensation from ω-Amino Ester

In another aspect, the disclosure provides methods of making a polyamide from a natural oil, the method comprising: providing C₁₋₈ alkyl esters of 9-decenoic acid; reacting esters of C₁₋₈ alkyl 9-decenoic acid with a brominating agent to form C₁₋₈ alkyl esters of 10-bromodecanoic acid; reacting C₁₋₈ alkyl esters of 10-bromodecanoic acid with an aminating agent to form C₁₋₈ alkyl esters of 10-aminodecanoic acid; converting the C₁₋₈ alkyl esters of 10-aminodecanoic acid to 10-aminodecanoic acid; and polymerizing 10-aminodecanoic acid to form a nylon-10 polymer.

In some embodiments, providing C₁₋₈ alkyl esters of 9-decenoic acid comprises deriving C₁₋₈ alkyl esters of 9-decenoic acid from a natural oil composition, such as a natural oil (as defined above) or any composition comprising a natural oil. Deriving the C₁₋₈ alkyl esters of 9-decenoic acid from a natural oil can be accomplished by any suitable means. For example, in some embodiments, deriving C₁₋₈ alkyl esters of 9-decenoic acid from a natural oil comprises: providing a natural oil composition comprising C₁₋₈ alkyl esters of unsaturated natural fatty acids; and reacting the C₁₋₈ alkyl esters of unsaturated natural fatty acids with a short-chain alpha-olefin in the presence of a metathesis catalyst to form C₁₋₈ alkyl esters of 9-decenoic acid and 1-decene. The aforementioned C₁₋₈ alkyl esters can be any suitable such esters. In some embodiments, the C₁₋₈ alkyl esters of unsaturated natural fatty acids are methyl esters of unsaturated natural fatty acids; the C₁₋₈ alkyl esters of 9-decenoic acid are methyl esters of 9-decenoic acid; the C₁₋₈ alkyl esters of 10-bromodecanoic acid are methyl esters of 10-bromodecanoic acid; and the C₁₋₈ alkyl esters of 10-aminodecanoic acid are methyl esters of 10-aminodecanoic acid.

Deriving the C₁₋₈ alkyl esters of 9-decenoic acid from a natural oil can be accomplished by any suitable means. In some embodiments, deriving C₁₋₈ alkyl esters of 9-decenoic acid from a natural oil comprises: providing a natural oil composition comprising glyceryl esters of unsaturated natural fatty acids; reacting the glyceryl esters of unsaturated natural fatty acids with a short-chain alpha-olefin in the presence of a metathesis catalyst to form glyceryl esters of 9-decenoic acid and 1-decene; and reacting the glyceryl esters of 9-decenoic acid with a C₁₋₈ monohydric alkanol to form C₁₋₈ esters of 9-decenoic acid.

In some other embodiments, deriving methyl esters of 9-decenoic acid from a natural oil comprises: providing a natural oil composition comprising glyceryl esters of unsaturated natural fatty acids; reacting the glyceryl esters of unsaturated natural fatty acids with a short-chain alpha-olefin in the presence of a metathesis catalyst to form glyceryl esters of 9-decenoic acid and 1-decene; and reacting the glyceryl esters of 9-decenoic acid with C₁₋₈ monohydric alkanol to form C₁₋₈ alkyl esters of 9-decenoic acid.

In the immediately aforementioned embodiments, any suitable C₁₋₈ monohydric alkanol can be used. In some embodiments, the C₁₋₈ monohydric alkanol is selected from the group consisting of: methanol, ethanol, propanol, isopropanol, butanol, sec-butanol, tert-butanol, pentanol, neopentanol, hexanol, heptanol, octanol, and 2-ethylhexanol. In some further such embodiments, the C₁₋₈ monohydric alkanol is methanol.

Any suitable unsaturated natural fatty acids can be used in the methods disclosed herein. In some embodiments, the unsaturated natural fatty acids are unsaturated fatty acids having a carbon-carbon double bond between the ninth and tenth carbon atoms counting from the ester group (including the carbon in the carbonyl of the ester). In some embodiments, the unsaturated natural fatty acids are selected from the group consisting of myristoleic acid, palmitoleic acid, oleic acid, elaidic acid, linoleic acid, linoelaidic acid, and α-linolenic acid. In some further embodiments, the unsaturated natural fatty acids are selected from the group consisting of oleic acid, linoleic acid, and α-linolenic acid.

In any of the aforementioned embodiments, reacting the unsaturated natural fatty acid esters with a short-chain alpha-olefin in the presence of a metathesis catalyst can be carried out by any suitable means. Principles of metathesis are discussed in greater detail in a subsequent subsection, and can be applied here. Any suitable short-chain alpha-olefin can be used. For example, in some embodiments, the short-chain alpha olefin is selected from the group consisting of: ethylene, propylene, 1-butene, isobutene, 1-pentene, 1-hexene, 1-heptene, 1-octene, and 1-nonene. In some further such embodiments, the short-chain alpha olefin is selected from the group consisting of: ethylene, propylene, and 1-butene. In some further such embodiments, the short-chain alpha olefin is selected from the group consisting of: ethylene or 1-butene.

The converting of the C₁₋₈ alkyl esters of 10-aminodecanoic acid to 10-aminodecanoic acid can be carried out by any suitable means. In some embodiments, converting the C₁₋₈ alkyl esters of 10-aminodecanoic acid to 10-aminodecanoic acid comprises hydrolyzing the C₁₋₈ alkyl esters of 10-aminodecanoic acid to form 10-aminodecanoic acid. In some other embodiments, converting the C₁₋₈ alkyl esters of 10-aminodecanoic acid to 10-aminodecanoic acid comprises: saponifying the C₁₋₈ alkyl esters of 10-aminodecanoic to form 10-aminodecanoate anions; and acidifying the 10-aminodecanoate anions to form 10-aminodecanoic acid. In either of the aforementioned embodiments, the C₁₋₈ alkyl esters of 9-decenoic acid are methyl 9-decenoate, the C₁₋₈ alkyl esters of 10-bromodecanoic acid are methyl 10-bromodecanoate, and the C₁₋₈ alkyl esters of 10-aminodecanoic acid are methyl 10-aminodecanoate.

The bromination can be carried by any suitable means. In some embodiments, the brominating agent is hydrobromic acid. Known methods of hydrobromination can be suitably modified to achieve the desired result, depending on the relevant scale.

The amination can be carried by any suitable means. In some embodiments, the aminating agent is ammonia. Known methods of reacting an alkyl bromide with ammonia can be suitably modified to achieve the desired result, depending on the relevant scale.

Methods of Making Polyamides by Alcoholic Condensation from ω-Amino Ester

In another aspect, the disclosure provides methods of making a polyamide from a natural oil, the method comprising: providing C₁₋₈ alkyl esters of 9-decenoic acid; reacting esters of C₁₋₈ alkyl 9-decenoic acid with a brominating agent to form C₁₋₈ alkyl esters of 10-bromodecanoic acid; reacting C₁₋₈ alkyl esters of 10-bromodecanoic acid with an aminating agent to form C₁₋₈ alkyl esters of 10-aminodecanoic acid; and polymerizing C₁₋₈ alkyl esters of 10-aminodecanoic acid to form a nylon-10 polymer.

In some embodiments, providing C₁₋₈ alkyl esters of 9-decenoic acid comprises deriving C₁₋₈ alkyl esters of 9-decenoic acid from a natural oil composition, such as a natural oil (as defined above) or any composition comprising a natural oil. Deriving the C₁₋₈ alkyl esters of 9-decenoic acid from a natural oil can be accomplished by any suitable means. For example, in some embodiments, deriving C₁₋₈ alkyl esters of 9-decenoic acid from a natural oil comprises: providing a natural oil composition comprising C₁₋₈ alkyl esters of unsaturated natural fatty acids; and reacting the C₁₋₈ alkyl esters of unsaturated natural fatty acids with a short-chain alpha-olefin in the presence of a metathesis catalyst to form C₁₋₈ alkyl esters of 9-decenoic acid and 1-decene. The aforementioned C₁₋₈ alkyl esters can be any suitable such esters. In some embodiments, the C₁₋₈ alkyl esters of unsaturated natural fatty acids are methyl esters of unsaturated natural fatty acids; the C₁₋₈ alkyl esters of 9-decenoic acid are methyl esters of 9-decenoic acid; the C₁₋₈ alkyl esters of 10-bromodecanoic acid are methyl esters of 10-bromodecanoic acid; and the C₁₋₈ alkyl esters of 10-aminodecanoic acid are methyl esters of 10-aminodecanoic acid.

Deriving the C₁₋₈ alkyl esters of 9-decenoic acid from a natural oil can be accomplished by any suitable means. In some embodiments, deriving C₁₋₈ alkyl esters of 9-decenoic acid from a natural oil comprises: providing a natural oil composition comprising glyceryl esters of unsaturated natural fatty acids; reacting the glyceryl esters of unsaturated natural fatty acids with a short-chain alpha-olefin in the presence of a metathesis catalyst to form glyceryl esters of 9-decenoic acid and 1-decene; and reacting the glyceryl esters of 9-decenoic acid with a C₁₋₈ monohydric alkanol to form C₁₋₈ esters of 9-decenoic acid.

In some other embodiments, deriving methyl esters of 9-decenoic acid from a natural oil comprises: providing a natural oil composition comprising glyceryl esters of unsaturated natural fatty acids; reacting the glyceryl esters of unsaturated natural fatty acids with a short-chain alpha-olefin in the presence of a metathesis catalyst to form glyceryl esters of 9-decenoic acid and 1-decene; and reacting the glyceryl esters of 9-decenoic acid with C₁₋₈ monohydric alkanol to form C₁₋₈ alkyl esters of 9-decenoic acid.

In the immediately aforementioned embodiments, any suitable C₁₋₈ monohydric alkanol can be used. In some embodiments, the C₁₋₈ monohydric alkanol is selected from the group consisting of: methanol, ethanol, propanol, isopropanol, butanol, sec-butanol, tert-butanol, pentanol, neopentanol, hexanol, heptanol, octanol, and 2-ethylhexanol. In some further such embodiments, the C₁₋₈ monohydric alkanol is methanol.

Any suitable unsaturated natural fatty acids can be used in the methods disclosed herein. In some embodiments, the unsaturated natural fatty acids are unsaturated fatty acids having a carbon-carbon double bond between the ninth and tenth carbon atoms counting from the ester group (including the carbon in the carbonyl of the ester). In some embodiments, the unsaturated natural fatty acids are selected from the group consisting of myristoleic acid, palmitoleic acid, oleic acid, elaidic acid, linoleic acid, linoelaidic acid, and α-linolenic acid. In some further embodiments, the unsaturated natural fatty acids are selected from the group consisting of oleic acid, linoleic acid, and α-linolenic acid.

In any of the aforementioned embodiments, reacting the unsaturated natural fatty acid esters with a short-chain alpha-olefin in the presence of a metathesis catalyst can be carried out by any suitable means. Principles of metathesis are discussed in greater detail in a subsequent subsection, and can be applied here. Any suitable short-chain alpha-olefin can be used. For example, in some embodiments, the short-chain alpha olefin is selected from the group consisting of: ethylene, propylene, 1-butene, isobutene, 1-pentene, 1-hexene, 1-heptene, 1-octene, and 1-nonene. In some further such embodiments, the short-chain alpha olefin is selected from the group consisting of: ethylene, propylene, and 1-butene. In some further such embodiments, the short-chain alpha olefin is selected from the group consisting of: ethylene or 1-butene.

The bromination can be carried by any suitable means. In some embodiments, the brominating agent is hydrobromic acid. Known methods of hydrobromination can be suitably modified to achieve the desired result, depending on the relevant scale.

The amination can be carried by any suitable means. In some embodiments, the aminating agent is ammonia. Known methods of reacting an alkyl bromide with ammonia can be suitably modified to achieve the desired result, depending on the relevant scale.

### Polymerization

The condensation polymerization, whether carried out by the elimination of water of a C₁₋₈ alkanol, can be carried out by any suitable means for the homopolymerization of w-amino acids or w-amino esters to make polyamides.

Derivation from Renewable Sources

As noted above, certain compounds employed in various aspects or embodiments disclosed herein can, in certain embodiments, be derived from renewable sources, such as from various natural oils or their derivatives. Any suitable methods can be used to make these compounds from such renewable sources.

Olefin metathesis provides one possible means to convert certain natural oil feedstocks into olefins and esters that can be used in a variety of applications, or that can be further modified chemically and used in a variety of applications. In some embodiments, a composition (or components of a composition) may be formed from a renewable feedstock, such as a renewable feedstock formed through metathesis reactions of natural oils and/or their fatty acid or fatty ester derivatives. When compounds containing a carbon-carbon double bond undergo metathesis reactions in the presence of a metathesis catalyst, some or all of the original carbon-carbon double bonds are broken, and new carbon-carbon double bonds are formed. The products of such metathesis reactions include carbon-carbon double bonds in different locations, which can provide unsaturated organic compounds having useful chemical properties.

A wide range of natural oils, or derivatives thereof, can be used in such metathesis reactions. Examples of suitable natural oils include, but are not limited to, vegetable oils, algae oils, fish oils, animal fats, tall oils, derivatives of these oils, combinations of any of these oils, and the like. Representative non-limiting examples of vegetable oils include rapeseed oil (canola oil), coconut oil, corn oil, cottonseed oil, olive oil, palm oil, peanut oil, safflower oil, sesame oil, soybean oil, sunflower oil, linseed oil, palm kernel oil, tung oil, jatropha oil, mustard seed oil, pennycress oil, camelina oil, hempseed oil, and castor oil. Representative non-limiting examples of animal fats include lard, tallow, poultry fat, yellow grease, and fish oil. Tall oils are by-products of wood pulp manufacture. In some embodiments, the natural oil or natural oil feedstock comprises one or more unsaturated glycerides (e.g., unsaturated triglycerides). In some such embodiments, the natural oil feedstock comprises at least 50% by weight, or at least 60% by weight, or at least 70% by weight, or at least 80% by weight, or at least 90% by weight, or at least 95% by weight, or at least 97% by weight, or at least 99% by weight of one or more unsaturated triglycerides, based on the total weight of the natural oil feedstock.

The natural oil may include canola or soybean oil, such as refined, bleached and deodorized soybean oil (i.e., RBD soybean oil). Soybean oil typically includes about 95 percent by weight (wt %) or greater (e.g., 99 wt % or greater) triglycerides of fatty acids. Major fatty acids in the polyol esters of soybean oil include but are not limited to saturated fatty acids such as palmitic acid (hexadecanoic acid) and stearic acid (octadecanoic acid), and unsaturated fatty acids such as oleic acid (9-octadecenoic acid), linoleic acid (9,12-octadecadienoic acid), and linolenic acid (9,12,15-octadecatrienoic acid).

Such natural oils, or derivatives thereof, contain esters, such as triglycerides, of various unsaturated fatty acids. The identity and concentration of such fatty acids varies depending on the oil source, and, in some cases, on the variety. In some embodiments, the natural oil comprises one or more esters of oleic acid, linoleic acid, linolenic acid, or any combination thereof. When such fatty acid esters are metathesized, new compounds are formed. For example, in embodiments where the metathesis uses certain short-chain alkenes, e.g., ethylene, propylene, or 1-butene, and where the natural oil includes esters of oleic acid, an amount of 1-decene and 1-decenoic acid (or an ester thereof), among other products, are formed.

In some embodiments, the natural oil can be subjected to various pre-treatment processes, which can facilitate their utility for use in certain metathesis reactions. Useful pre-treatment methods are described in United States Patent Application Publication Nos. 2011/0113679, 2014/0275595, and 2014/0275681, all three of which are hereby incorporated by reference as though fully set forth herein.

In some embodiments, after any optional pre-treatment of the natural oil feedstock, the natural oil feedstock is reacted in the presence of a metathesis catalyst in a metathesis reactor. In some other embodiments, an unsaturated ester (e.g., an unsaturated glyceride, such as an unsaturated triglyceride) is reacted in the presence of a metathesis catalyst in a metathesis reactor. These unsaturated esters may be a component of a natural oil feedstock, or may be derived from other sources, e.g., from esters generated in earlier-performed metathesis reactions.

The conditions for such metathesis reactions, and the reactor design, and suitable catalysts are as described below with reference to the metathesis of the olefin esters. That discussion is incorporated by reference as though fully set forth herein.

### Olefin Metathesis

In some embodiments, one or more of the unsaturated monomers can be made by metathesizing a natural oil or natural oil derivative. The terms “metathesis” or “metathesizing” can refer to a variety of different reactions, including, but not limited to, cross-metathesis, self-metathesis, ring-opening metathesis, ring-opening metathesis polymerizations (“ROMP”), ring-closing metathesis (“RCM”), and acyclic diene metathesis (“ADMET”). Any suitable metathesis reaction can be used, depending on the desired product or product mixture.

In some embodiments, after any optional pre-treatment of the natural oil feedstock, the natural oil feedstock is reacted in the presence of a metathesis catalyst in a metathesis reactor. In some other embodiments, an unsaturated ester (e.g., an unsaturated glyceride, such as an unsaturated triglyceride) is reacted in the presence of a metathesis catalyst in a metathesis reactor. These unsaturated esters may be a component of a natural oil feedstock, or may be derived from other sources, e.g., from esters generated in earlier-performed metathesis reactions. In certain embodiments, in the presence of a metathesis catalyst, the natural oil or unsaturated ester can undergo a self-metathesis reaction with itself.

In some embodiments, the metathesis comprises reacting a natural oil feedstock (or another unsaturated ester) in the presence of a metathesis catalyst. In some such embodiments, the metathesis comprises reacting one or more unsaturated glycerides (e.g., unsaturated triglycerides) in the natural oil feedstock in the presence of a metathesis catalyst. In some embodiments, the unsaturated glyceride comprises one or more esters of oleic acid, linoleic acid, linoleic acid, or combinations thereof. In some other embodiments, the unsaturated glyceride is the product of the partial hydrogenation and/or the metathesis of another unsaturated glyceride (as described above).

The metathesis process can be conducted under any conditions adequate to produce the desired metathesis products. For example, stoichiometry, atmosphere, solvent, temperature, and pressure can be selected by one skilled in the art to produce a desired product and to minimize undesirable byproducts. In some embodiments, the metathesis process may be conducted under an inert atmosphere. Similarly, in embodiments where a reagent is supplied as a gas, an inert gaseous diluent can be used in the gas stream. In such embodiments, the inert atmosphere or inert gaseous diluent typically is an inert gas, meaning that the gas does not interact with the metathesis catalyst to impede catalysis to a substantial degree. For example, non-limiting examples of inert gases include helium, neon, argon, methane, and nitrogen, used individually or with each other and other inert gases.

The reactor design for the metathesis reaction can vary depending on a variety of factors, including, but not limited to, the scale of the reaction, the reaction conditions (heat, pressure, etc.), the identity of the catalyst, the identity of the materials being reacted in the reactor, and the nature of the feedstock being employed. Suitable reactors can be designed by those of skill in the art, depending on the relevant factors, and incorporated into a refining process such, such as those disclosed herein.

The metathesis reactions disclosed herein generally occur in the presence of one or more metathesis catalysts. Such methods can employ any suitable metathesis catalyst. The metathesis catalyst in this reaction may include any catalyst or catalyst system that catalyzes a metathesis reaction. Any known metathesis catalyst may be used, alone or in combination with one or more additional catalysts. Examples of metathesis catalysts and process conditions are described in US 2011/0160472, incorporated by reference herein in its entirety, except that in the event of any inconsistent disclosure or definition from the present specification, the disclosure or definition herein shall be deemed to prevail. A number of the metathesis catalysts described in US 2011/0160472 are presently available from Materia, Inc. (Pasadena, Calif.).

In some embodiments, the metathesis catalyst includes a Grubbs-type olefin metathesis catalyst and/or an entity derived therefrom. In some embodiments, the metathesis catalyst includes a first-generation Grubbs-type olefin metathesis catalyst and/or an entity derived therefrom. In some embodiments, the metathesis catalyst includes a second-generation Grubbs-type olefin metathesis catalyst and/or an entity derived therefrom. In some embodiments, the metathesis catalyst includes a first-generation Hoveyda-Grubbs-type olefin metathesis catalyst and/or an entity derived therefrom. In some embodiments, the metathesis catalyst includes a second-generation Hoveyda-Grubbs-type olefin metathesis catalyst and/or an entity derived therefrom. In some embodiments, the metathesis catalyst includes one or a plurality of the ruthenium carbene metathesis catalysts sold by Materia, Inc. of Pasadena, Calif. and/or one or more entities derived from such catalysts. Representative metathesis catalysts from Materia, Inc. for use in accordance with the present teachings include but are not limited to those sold under the following product numbers as well as combinations thereof: product no. C823 (CAS no. 172222-30-9), product no. C848 (CAS no. 246047-72-3), product no. C601 (CAS no. 203714-71-0), product no. C627 (CAS no. 301224-40-8), product no. C571 (CAS no. 927429-61-6), product no. C598 (CAS no. 802912-44-3), product no. C793 (CAS no. 927429-60-5), product no. C801 (CAS no. 194659-03-9), product no. C827 (CAS no. 253688-91-4), product no. C884 (CAS no. 900169-53-1), product no. C833 (CAS no. 1020085-61-3), product no. C859 (CAS no. 832146-68-6), product no. C711 (CAS no. 635679-24-2), product no. C933 (CAS no. 373640-75-6).

In some embodiments, the metathesis catalyst includes a molybdenum and/or tungsten carbene complex and/or an entity derived from such a complex. In some embodiments, the metathesis catalyst includes a Schrock-type olefin metathesis catalyst and/or an entity derived therefrom. In some embodiments, the metathesis catalyst includes a high-oxidation-state alkylidene complex of molybdenum and/or an entity derived therefrom. In some embodiments, the metathesis catalyst includes a high-oxidation-state alkylidene complex of tungsten and/or an entity derived therefrom. In some embodiments, the metathesis catalyst includes molybdenum (VI). In some embodiments, the metathesis catalyst includes tungsten (VI). In some embodiments, the metathesis catalyst includes a molybdenum- and/or a tungsten-containing alkylidene complex of a type described in one or more of (a) Angew. Chem. Int. Ed. Engl., 2003, 42, 4592-4633; (b) Chem. Rev., 2002, 102, 145-179; and/or (c) Chem. Rev., 2009, 109, 3211-3226, each of which is incorporated by reference herein in its entirety, except that in the event of any inconsistent disclosure or definition from the present specification, the disclosure or definition herein shall be deemed to prevail.

In certain embodiments, the metathesis catalyst is dissolved in a solvent prior to conducting the metathesis reaction. In certain such embodiments, the solvent chosen may be selected to be substantially inert with respect to the metathesis catalyst. For example, substantially inert solvents include, without limitation: aromatic hydrocarbons, such as benzene, toluene, xylenes, etc.; halogenated aromatic hydrocarbons, such as chlorobenzene and dichlorobenzene; aliphatic solvents, including pentane, hexane, heptane, cyclohexane, etc.; and chlorinated alkanes, such as dichloromethane, chloroform, dichloroethane, etc. In some embodiments, the solvent comprises toluene.

In other embodiments, the metathesis catalyst is not dissolved in a solvent prior to conducting the metathesis reaction. The catalyst, instead, for example, can be slurried with the natural oil or unsaturated ester, where the natural oil or unsaturated ester is in a liquid state. Under these conditions, it is possible to eliminate the solvent (e.g., toluene) from the process and eliminate downstream olefin losses when separating the solvent. In other embodiments, the metathesis catalyst may be added in solid state form (and not slurried) to the natural oil or unsaturated ester (e.g., as an auger feed).

The metathesis reaction temperature may, in some instances, be a rate-controlling variable where the temperature is selected to provide a desired product at an acceptable rate. In certain embodiments, the metathesis reaction temperature is greater than −40° C., or greater than −20° C., or greater than 0° C., or greater than 10° C. In certain embodiments, the metathesis reaction temperature is less than 200° C., or less than 150° C., or less than 120° C. In some embodiments, the metathesis reaction temperature is between 0° C. and 150° C., or is between 10° C. and 120° C.

### EXAMPLES

The following examples show certain illustrative embodiments of the compounds, compositions, and methods disclosed herein. These examples are not to be taken as limiting in any way. Nor should the examples be taken as expressing any preferred embodiments, or as indicating any direction for further research.

### Example 1—Synthesis of 9-Decenoic Acid

Methyl 9-decenoate (DAME) was obtained from the butenolysis (1-butene) of palm oil followed by separation of glycerides from alkenes, transesterification of the glycerides with methanol, and separation of DAME from other esters.

A 5-L 5-necked round-bottom flask was fitted with a mechanical stirrer, an additional funnel, a condenser, a thermocouple, and a stopper. The flask was charged with 1106 g of DAME, 540 mL of water, and 300 mL of isopropyl alcohol. Through the headspace of the flask, nitrogen gas was passed for 15 minutes. An aqueous solution of potassium hydroxide (10 M, 660 mL) was added over a period of 5 minutes. The mixture slowly became homogeneous and the temperature peaked at 55° C. The reaction mixture was stirred for four hours at a temperature of about 30° C. The mixture was placed in a water bath. Concentrated aqueous hydrochloric acid (37%, 600 mL) was added in portions over a period of one hour, while the temperature was maintained below 40° C. until the pH was 1-2. The organic layer was washed with saturated NaCl (3×250 mL), dried over Na₂SO₄, vacuum filtered, and concentrated under reduced pressure. The product was distilled (2 torr, 140° C.) to obtain 957 g of product as a colorless liquid.

### Example 2—Synthesis of 10-Bromodecanoic acid

A 500-mL, 3-necked round-bottom flask was fitted with a thermocouple, a gas dispersion tube, and a magnetic stir bar. The experimental apparatus also included dry traps that separated the reaction mixture from the hydrobromic acid lecture bottle and scrubber (water). The reaction flask was charged with 50 g of 9-decenoic acid from Example 1, 125 mL of toluene, and 0.75 g of benzoyl peroxide. The solution was placed in an ice-water bath and cooled to about 5° C. Hydrobromic acid was bubbled through the mixture over the course of about 1.25 hours while maintaining the temperature between 5-15° C. No exotherm was observed during the during the last few minutes of addition, and 33.3 g of HBr was absorbed. The mixture was washed with 50 mL each water and then brine. The organic phase was dried over magnesium sulfate, filtered, and concentrated on a rotovap (10 torr, 55° C.). The resulting oil was cooled to give an oily solid (73 g). The material was treated with 50 mL of hexane and filtered. The filter cake was washed with 2×20 mL hexane and dried in air to give a solid white product (30 g). The mother liquor was cooled in an ice bath for 2 hours to produce a second crop of product (17.5 g). The two crops were combined to give 47.5 g of product. The yield was 64%. The chemical shifts for the ¹H NMR were as follows (relative to TMS): 1.28-1.39 (m, 10H), 1.58-1.61 (m, 2H), 1.79-1.86 (m, 2H), 2.30-2.34 (m, 2H), 3.36-3.39 (m, 2H), 11.45-11.50 (b, 1H).

### Example 3—Synthesis of 10-Aminodecanoic Acid

A 250-mL round-bottom flask was charged with 5.0 g of 10-bromodecanoic acid from Example 2 and 100 mL of ammonium hydroxide (28% in water). The suspension was stirred at 20° C. for 3 hours. The suspension was heated to about 45° C. for 15 minutes. The mixture was then stirred at ambient temperature for 2 hours, then filtered. The filter cake was washed with water and 50% isopropanol. The resulting solid was dried in vacuo to give a white powder (about 1 g). The chemical shifts for the ¹H NMR were as follows (relative to TMS): 1.35 (s, 10H), 1.63-1.70 (m, 4H), 2.05-2.07 (m, 2H), 2.36-2.40 (m, 2H), 3.03-3.07 (m, 2H), 11.75 (approx., s, 1H).

### Example 4—Polymerization

Several mg of the 10-1 aminodecanoic acid from Example 3 was polymerized via melt polymerization at 220° C. An opaque solid was obtained.

1. A method of making a polyamide from a natural oil, the method comprising:
providing 9-decenoic acid, wherein providing 9-decenoic acid comprises deriving 9-decenoic acid from a natural oil composition;
reacting 9-decenoic acid with a brominating agent to form 10-bromodecanoic acid;
reacting 10-bromodecanoic acid with an aminating agent to form 10-aminodecanoic acid; and
polymerizing 10-aminodecanoic acid to form a polyamide-10 polymer.

2. The method of claim 1, wherein deriving 9-decenoic acid from a natural oil comprises:
providing a natural oil composition comprising unsaturated natural fatty acid esters;
reacting the unsaturated natural fatty acid esters with a short-chain alpha-olefin in the presence of a metathesis catalyst to form 9-decenoic acid esters and 1-decene; and
converting the 9-decenoic acid esters to 9-decenoic acid.

3. The method of claim 2, wherein the unsaturated natural fatty acid esters are C₁₋₈ alkyl esters of unsaturated natural fatty acids, and wherein the 9-decenoic acid esters are C₁₋₈ alkyl esters of 9-decenoic acid.

4. The method of claim 3, wherein the unsaturated natural fatty acid esters are methyl esters of unsaturated natural fatty acids, and wherein the 9-decenoic acid esters are methyl esters of 9-decenoic acid.

5. The method of claim 2, wherein the unsaturated natural fatty acid esters are glyceryl esters of unsaturated natural fatty acids, and wherein the 9-decenoic acid esters are glyceryl esters of 9-decenoic acid.

6. The method of claim 2, wherein the unsaturated natural fatty acids are selected from the group consisting of myristoleic acid, palmitoleic acid, oleic acid, elaidic acid, linoleic acid, linoelaidic acid, and α-linolenic acid.

7. The method of claim 6, wherein the unsaturated natural fatty acids are selected from the group consisting of oleic acid, linoleic acid, and α-linolenic acid.

8. The method of claim 2, wherein converting the 9-decenoic acid esters to 9-decenoic acid comprises hydrolyzing the 9-decenoic acid esters to form 9-decenoic acid.

9. The method of claim 2, wherein converting the 9-decenoic acid esters to 9-decenoic acid comprises:
saponifying the 9-decenoic acid esters to form 9-decenoate anions; and
acidifying the 9-decenoate anions to form 9-decenoic acid.

10. The method of claim 5, wherein converting the 9-decenoic acid esters to 9-decenoic acid comprises:
reacting the glyceryl esters of 9-decenoic acid with a C₁₋₈ monohydric alkanol to form C₁₋₈ esters of 9-decenoic acid; and
converting the C₁₋₈ esters of 9-decenoic acid to 9-decenoic acid.

11. The method of claim 10, wherein the C₁₋₈ monohydric alkanol is selected from the group consisting of: methanol, ethanol, propanol, isopropanol, butanol, sec-butanol, tert-butanol, pentanol, neopentanol, hexanol, heptanol, octanol, and 2-ethylhexanol.

12. The method of claim 11, wherein the C₁₋₈ monohydric alkanol is methanol.

13. The method of claim 10, wherein the unsaturated natural fatty acids are selected from the group consisting of myristoleic acid, palmitoleic acid, oleic acid, elaidic acid, linoleic acid, linoelaidic acid, and α-linolenic acid.

14. The method of claim 13, wherein the unsaturated natural fatty acids are selected from the group consisting of oleic acid, linoleic acid, and α-linolenic acid.

15. The method of claim 10, wherein converting the C₁₋₈ esters of 9-decenoic acid to 9-decenoic acid comprises hydrolyzing the C₁₋₈ esters of 9-decenoic acid to form 9-decenoic acid.

16. The method of claim 10, wherein converting the C₁₋₈ esters of 9-decenoic acid to 9-decenoic acid comprises:
saponifying the C₁₋₈ esters of 9-decenoic acid to form 9-decenoate anions; and
acidifying the 9-decenoate anions to form 9-decenoic acid.

17. The method of claim 2, wherein the short-chain alpha olefin is selected from the group consisting of: ethylene, propylene, 1-butene, isobutene, 1-pentene, 1-hexene, 1-heptene, 1-octene, and 1-nonene.

18. The method of claim 17, wherein the short-chain alpha olefin is selected from the group consisting of: ethylene, propylene, and 1-butene.

19. The method of claim 1, wherein the brominating agent is hydrobromic acid.

20. The method of claim 1, wherein the aminating agent is ammonia.

21-55. (canceled)
